(19) United States
(12) Patent Application Publication
(10) Pub. No.: US 2025/0346851 A1
(43) Pub. Date: Nov. 13, 2025

(54) A METHOD FOR PRODUCTION OF STABILIZED CULTURES

(71) Applicant: Chr. Hansen A/S, Hoersholm (DK)

(72) Inventors: Zuzana MLADENOVSKA, Hoersholm (DK); Surender Kumar DHAYAL, Hoersholm (IN)

(73) Assignee: Chr. Hansen A/S, Hoersholm (DK)

(21) Appl. No.: 18/869,389

(22) PCT Filed: May 31, 2023
(86) PCT No.: PCT/EP2023/064550

§ 371 Date: Nov. 26, 2024

(30) Foreign Application Priority Data
Jun. 1, 2022 (EP) 22176753.6

(51) Int. Cl. C12N 1/04; C12N 1/20

(52) U.S. Cl. CPC C12N 1/04; C12N 1/20

(57) ABSTRACT

The present invention relates to the field of frozen or dry compositions for prokaryotes, in particular fermentative bacteria such as lactic acid bacteria, a method for preparing frozen or dry prokaryotic compositions with improved stability and compositions which may be prepared by said method.

The present invention relates to the field of frozen or dry compositions for certain bacteria, in particular fermentative bacteria such as lactic acid bacteria, a method for preparing frozen or dry bacterial compositions and compositions which may be prepared by said method.

### BACKGROUND OF THE INVENTION

Fermentative bacteria are anaerobic bacteria in the metabolism of which an organic compound (instead of oxygen) is the terminal electron (or hydrogen) acceptor. Based on the pattern of products formed in fermentations, bacteria are classified as homofermentative and heterofermentative. Lactic acid bacteria (LAB) with homofermentative metabolism, produce lactic acid as the major or sole product of sugar fermentation. Examples of homofermentative lactic acid bacteria are species Lactococcus lactis, Lactobacillus delbrueckii subsp. bulgaricus or Streptococcus thermophilus. Heterofermentative bacteria produce various products from fermentation of sugars and the end products depends on the type of sugar served in fermentation. Heterofermentative lactic acid bacteria, such Oenococcus, Leuconostoc and some Lactobacillus species, such as Lactobacillus reuteri, ferment sugars in addition to lactate, CO₂ and ethanol, also to acetate and polyols. The present invention is applicable to both types of fermentative bacteria.

Fermentative bacteria are involved in numerous industrially relevant processes. For instance, bacterial cultures, in particular cultures of bacteria that are generally classified as LAB, are essential in the making of all fermented milk products, cheese and butter. Cultures of such bacteria may be referred to as starter cultures and they impart specific features to various dairy products by performing a number of functions.

Many lactic acid bacteria are known to have probiotic properties (i.e. they have a beneficial health effect on humans and animals when ingested). Probiotics are widely applied in dry form. In most cases, it is imperative that the microorganisms remain viable after prolonged storage of dried products, in order for these to impart their beneficial effect.

Since it is well known that bacteria can easily lose viability upon exposure to various stresses, it is a general practice in industrial production of bacterial cultures to use protectants. These protectants are supposed to protect cells during different steps of a production process and later on during shelf storage of dried bacteria. Bacteria that are to be frozen or dried, for example spray-dried, freeze-dried, vacuum-dried, are mixed as a cell suspension with protectants and then processed in a sequence of various technological steps. The role of the protectant is to protect the bacterial cell composition during freezing (so called cryo-protectants), drying or freeze-drying (so called lyo-protectants). However, certain damage of cells during these processes cannot be avoided (Coulibaly et al. (2018) ARRB 24 (4): 1-15).

Bacterial products can also be formulated as frozen products. For example, commercial starter cultures may be distributed as frozen cultures. Highly concentrated frozen cultures, particularly when prepared as pellets, are commercially very useful since such cultures can be inoculated directly into the fermentation medium (e.g. milk or meat) without intermediate transfer. In other words, such highly concentrated frozen cultures comprise bacteria in an amount that makes in-house bulk starter cultures at the end-users superfluous. A “bulk starter” is defined herein as a starter culture propagated at the food processing plant for inoculation into the fermentation medium. Highly concentrated cultures may be referred to as direct vat set (DVS)-cultures. In order to comprise sufficient bacteria to be used as a DVS-culture at the end-users, a concentrated frozen culture generally has to have a weight of at least 50 g and a content of viable bacteria of at least 109 colony forming units (CFU) per g. WO 2005/080548 (Chr. Hansen) discloses pellet-frozen lactic acid bacteria (LAB) cultures that are stabilised with, for example, a mixture of trehalose and sucrose and do not form clumps when stored.

The prior art discloses maintaining the cell culture at 4° C. during all intermediate steps of the process, including during the step of formulating the cell concentrate with protectant, with the aims of limiting the cell degradation reactions. (Fonseca et al. (2015) Chapter 24: Freeze-Drying of Lactic Acid Bacteria in Wolkers & Oldenhof (Eds), Cryopreservation and Freeze-drying Protocols, Third edition).

In prior art processes, a concentrated bacterial culture is obtained by known methods of culturing the bacteria in a growth medium and then concentrating the culture, for example by centrifugation, with the bacteria being separated from the growth medium. The concentrated culture is then admixed with the desired preservative(s) and, shortly thereafter, the resulting mixture is frozen or dried.

The microbial cell surface has a very complex composition and it plays a key role in interactions between microorganisms and the surrounding environment (Burgain J, et al (2014) Advances in Colloid and Interface Science 213, 21-35).

The cell wall of Gram-positive bacteria consists of a peptidoglycan layer with embedded teichoic, lipoteichoic acid and cell wall polysaccharides. The peptidoglycan layer can be covered by a proteinaceous S-layer and decorated by various polysaccharides (Zeidan et al 2017, FEMS Microbiology Reviews 41: 168-200). The surface of Gram-negative bacteria is different. It is made of capsular polysaccharides which are decorated with various polymeric substances such as carbohydrates, lipo-oligosaccharides and lipopolysaccharides. This complex composition of cell surface can be captured by physicochemical analyses such as measurement of cell surface interactions by hydrophobicity analysis and cell surface charge determined by zeta potential. Particularly, the combination of these two assays with advanced microscopy techniques has contributed to a more profound characterization of the cell wall of lactic acid bacteria (Schär-Zammaretti and Ubbink (2003) Biophysical Journal 85, 4076-4092). The hydrophobicity analysis, originally developed by Rosenberg et al. (1980, FEMS Microbiology Letters 9, 29-33) as a measurement of bacterial cell adherence to liquid hydrocarbon, was refined by Schär-Zammaretti and Ubbink (op. cit.) into determination of interfacial adhesion curves, reflecting partitioning of bacteria from aqueous phase to hexadecane in organic phase. From the pattern of the interfacial adhesion curves and zeta potential it is possible to differentiate between the primary constituents of the cell surface. The presence of surface proteins was found to be correlated with elevated isoelectric point and high hydrophobicity of surface. Teichoic acid made the surface hydrophobic and strongly negatively charged. A high abundance of polysaccharides rendered the cell surface hydrophilic and weakly charged.

The characterization of cell surface properties and links to the growth conditions of bacteria have been the subjects of numerous studies (Schär-Zammaretti et al (2005) AEM 71, 8165-8173; and Millsap K-W et al (1996) J. Microbiol. Methods 27, 239-242). These studies demonstrated that the composition of the growth medium in a fermentation process had a significant impact not only on the cell yield, but also on the cell surface properties of lactic acid bacteria.

In industrial processes for the production of beneficial bacteria, it is important that bacterial cells exhibit a high degree of robustness and maintain viability after fermentation, during several steps in the downstream processes. The link between the physicochemical characteristics of cells and cell survival in the downstream process has been described solely in the study of Zupancic et al. (Pharmaceutics 2019, 11, 483; doi:10.3390/pharmaceutics11090483). In this work it was demonstrated that lactic acid bacteria with hydrophobic cell surface survived better the process of electrospinning than bacteria with hydrophilic cell surface. However, Shakirova et al. J Ind Microbiol Biotechnol (2013) 40:85-93, showed an inverse relationship between cell surface hydrophobicity and survival of cells subjected to subsequent conditions like long-term storage.

### SUMMARY OF THE INVENTION

The present invention is derived from the unexpected observation that cells with a certain cell surface hydrophobicity show improved tolerance to long-term storage, if certain protectant compounds were added in the downstream processing. The invention will now be defined in more detail.

The invention provides a method of preparing a frozen, dried or freeze-dried product comprising an asporogenous prokaryote, the method comprising the steps of:
i. growing the prokaryote cells by fermentation;
ii. concentrate the cells by separation of fermentation broth;
iii. combining cell concentrate with a medium containing a protective compound, to obtain a preprocessing composition;
iv. holding the preprocessing composition under conditions not inducing metabolic activity
v. preserving the composition by
   (a) freezing the composition to form a frozen prokaryote product;
   (b) drying the product to form a dried prokaryote product; or
   (c) freezing the composition to form a frozen prokaryote intermediate product and then lyophilising the intermediate product to form a freeze-dried prokaryote product,
wherein the hydrophobicity of the cell surface of the cells is in the product is at least 40%, as measured by the MATH method at 22° C. and expressed as [(Initial OD₆₀₀−Final OD₆₀₀)/Initial OD₆₀₀]*100 when measured with a ϕ[V_{H}/V_{B}] at at least one point between 0.1 and 1.0 and the initial OD₆₀₀ (nm) is 0.5 and wherein the potency of product is 1E+08 to 1E+13 CFU/g.

In terms of an increase in hydrophobicity, the starting value and the finishing value should be measured at the same ϕ [V_{H}/V_{B}] value.

A fermentation broth will usually have 5E+08 to 1E+11 total cells/g fermentation broth, where ‘total cells’ means viable and non-viable cells and the weight of the fermentation broth includes the cells suspended in it. The concentration of cells in a liquid can be measured by standard techniques such as the Petroff Hausser counting chamber method or flow cytometry.

A concentrated culture (“cell concentrate”) is generally formed by separating the cells from a fermentation broth with a concentration factor of 2× to 90×, typically 5× to 60×, for example 10× to 50× or 20× to 40×. The total concentration of cells in the cell concentrate will therefore be in the range 1E+09 to 9E+12 prokaryote cells/g, preferably 2.5E+09 to 3E+12 prokaryote cells/g, 1.3E+10 to 2E+12 prokaryote cells/g, 2E+10 to 1.3E+12 prokaryote cells/g, 3E+10 to 2.5E+11 prokaryote cells/g, or 4.5E+10 to 1E+11 prokaryote cells/g.

The protective compound may, for example, be one or more of: a monosaccharide such as glucose, fructose, galactose or mannose; a disaccharide such as sucrose, trehalose, maltose or lactose; a sugar alcohol such as inositol; a trisaccharide such as maltotriose or raffinose; an oligosaccharide such as a fructooligosaccharide or such as a maltodextrin with DE 3-20; a polysaccharide such as starch or inulin; a cryoprotectant and/or a lyoprotectant and/or a storage stabiliser, such as gum arabic, a maltodextrin, starch, pectin, cellulose, xylan, or a polyol such as glycerol, sucrose, trehalose or maltose, a protein such as gelatin, a peptide such as are supplied by yeast extract, an amino acid such as proline or a sugar alcohol such as sorbitol, mannitol or inositol, an antioxidant, such as sodium ascorbate, sodium citrate.

Preferably, step (ii) lasts for 0.25 to 16 hours and is best carried out at 4° C. to 20° C., preferably below 10° C.

Preferably, the frozen prokaryote product or the frozen prokaryote intermediate product has a dry weight ratio of the final protectant to cell concentrate of between 10:1 and 0.1:1, preferably between 3:1 and 0.5:1 and most preferably between 2:1 and 1:1.

The method of the invention is widely applicable. The prokaryote may be a fermentative bacterium
from the phylum Firmicutes, such as:
a lactic acid bacterium (LAB), preferably of a genus selected from the group consisting of Streptococcus (such as Streptococcus thermophilus), Lactococcus (such as Lactococcus lactis), Oenococcus (such as Oenococcus oeni), Leuconostoc (such as species Leuconostoc mesenteroides, Leuconostoc pseudomesenteroides), Lactobacillus, Limosilactobacillus, Lacticaseibacillus, Ligilactobacillus, Lacticasei-bacillus, Lacticaseibacillus, Lactiplantibacillus, Limosilactobacillus, Ligilactobacillus, Lentilactobacillus, Latilactobacillus, Companilacto-bacillus, Latilactobacillus and Lactiplantibacillus; Eubacterium (such as Eubacterium limosum, Eubacterium aggregans, Eubacterium barkeri, Eubacterium lentum), Roseburia (Roseburia intestinalis, Roseburia hominis, Roseburia inulinivorans, Roseburia faecis and Roseburia cecicola), Faecalibacterium (such as species Faecalibacterium prausnitzii), Anaerostipes (such as Anaerostipes cacccae), Anaero-butyricum (such as species Anaerobutyricum hallii, Anaerobutyricum soehngenii)
from the phylum Actinobacteria, such as genus Bifidobacterium (such as species Bifidobacterium animalis, Bifidobacterium longum, Bifido-bacterium adolescentis, Bifidobacterium breve), genus Propioni-bacterium (such as species Propionibacterium freudenreichii), Cuti-bacterium (such as Cutibacteriun acnes)
from the phylum Bacteroidetes, such as genera Bacteroides (such as species Bacteroides fragilis, Bacteroides xylanisolvens), genus Prevotella (such as species Prevotella copri) or Alistipes
or from the phylum Verrucomicrobia, such as an Akkermansia (such as species Akkermansia muciniphila).

In particular, the prokaryote can be one or more of: Limosilactobacillus reuteri, Lacticaseibacillus rhamnosus, Ligilactobacillus salivarius, Lacticaseibacillus casei, Lacticaseibacillus paracasei subsp. paracasei, Lactiplantibacillus plantarum subsp. plantarum, Limosilactobacillus fermentum, Ligilactobacillus animalis, Lentilactobacillus buchneri, Latilactobacillus curvatus, Companilactobacillus futsaii, Latilactobacillus sakei subsp. sakei, Lactiplantibacillus pentosus, Levilactobacillus brevis, Lactobacillus delbrueckii subsp. bulgaricus, Lactobacillus delbrueckii subsp. lactis, Lactobacillus gasseri, Lactobacillus johnsonii, Lactobacillus helveticus and Lactobacillus acidophilus, Lactobacillus jensenii, and Lactobacillus iners.

The invention furthermore provides a frozen or dried product comprising an asporogenous prokaryote, obtainable by the method described above.

The potency of the frozen or dried product can be 1E+08-1E+13 CFU/g.

The method is applicable to vegetative cells of prokaryotic microorganisms from the domain Bacteria and Archaea. The invention relates to a broad spectrum of non-sporulating microorganisms used in food- and feed-producing industries, agriculture, medicine, for production of biofuels and biobased chemicals.

Non-spore-forming bacteria can be identified within the phyla Firmicutes, Actinobacteria and Bacteroidetes. The invention is particularly applicable to homo- and heterofermentative lactic acid bacteria in the Firmicutes phylum, and to bifidobacteria and propionibacteria in the Actinobacteria phylum. The invention is also applicable to obligate anaerobes of the class Clostridia in the Firmicutes phylum, such as fermentative, butyrate-producing bacteria of the genera Roseburia (e.g. Roseburia hominis and Roseburia inulinivorans), Anaerobutyricum hallii, Anaerobutyricum soehngenii), Eubacterium (e.g. Eubacterium limosum), Anaerostipes (e.g. Anaerostipes caccae), and Faecalibacterium (e.g. F. prausnitzii) which represent the core microbiota of human intestinal tract and are candidates for next generation of probiotics.

The industrially most useful lactic acid bacteria are found among Lactococcus species, Streptococcus species, Enterococcus species, Lactobacillus species (including all those that were classed as Lactobacillus until 2020), Leuconostoc species, Oenococcus, Bifidobacterium species, Propionibacterium and Pediococcus species. Accordingly, in a preferred embodiment the lactic acid bacteria are selected from the group consisting of these lactic acid bacteria.

The lactic acid bacteria are preferably of a genus selected from the group consisting of Lactobacillus, Limosilactobacillus, Lacticaseibacillus, Ligilactobacillus, Lacticasei-bacillus, Lacticaseibacillus, Lactiplantibacillus, Limosilactobacillus, Ligilactobacillus, Lentilactobacillus, Latilactobacillus, Companilactobacillus, Latilactobacillus and Lacti-plantibacillus. In particular, they can be Limosilactobacillus reuteri, Lacticaseibacillus rhamnosus, Ligilactobacillus salivarius, Lacticaseibacillus casei, Lacticaseibacillus paracasei subsp. paracasei, Lactiplantibacillus plantarum subsp. plantarum, Limo-silactobacillus fermentum, Ligilactobacillus animalis, Lentilactobacillus buchneri, Latilactobacillus curvatus, Companilactobacillus futsaii, Latilactobacillus sakei subsp. sakei, and/or Lactiplantibacillus pentosus. Others include Lactococcus lactis subsp. lactis, Lactococcus lactis subsp. cremoris, Leuconostoc lactis, Leuconostoc mesenteroides subsp. cremoris, Pediococcus pentosaceus, Lactococcus lactis subsp. lactis biovar. diacetylactis, Streptococcus thermophilus, Enterococcus, such as Enterococcus faecium, Bifidobacterium animalis subsp. lactis, Bifidobacterium animalis subsp. animalis, Bifidobacterium longum, Bifidobacterium adolescentis, Bifidobacterium breve, Lactobacillus helveticus, Lactobacillus fermentum, Lactobacillus salivarius, Lactobacillus delbrueckii subsp. bulgaricus and Lactobacillus acidophilus.

The composition may comprise one or more strains of lactic acid bacteria which may be selected from the group comprising: BB-12® (Bifidobacterium animalis subsp lactis BB-12®), DSM 15954; ATCC 29682, ATCC 27536, DSM 13692, DSM 10140, LA-5® (Lactobacillus acidophilus LA-5®), DSM 13241, LGG® (Lactobacillus rhamnosus LGG®), ATCC 53103, GR-1® (Lactobacillus rhamnosus GR-1®), ATCC 55826, RC-14® (Lactobacillus reuteri RC-14®), ATCC 55845, L. casei 431® (Lactobacillus paracasei subsp. paracasei L. casei 431®), ATCC 55544, F19® (Lactobacillus paracasei F19®), LMG-17806, TH-4® (Streptococcus thermophilus TH-4®©), DSM 15957, PCC® (Lactobacillus fermentum PCC®), NM02/31074, and LP-33® (Lactobacillus paracasei subsp. paracasei LP-33®), CCTCC M204012.

The LAB culture may be a “mixed lactic acid bacteria (LAB) culture” or a “pure lactic acid bacteria (LAB) culture”. The term “mixed lactic acid bacteria (LAB) culture”, or “LAB” culture, denotes a mixed culture that comprises two or more different LAB species. The term a “pure lactic acid bacteria (LAB) culture” denotes a pure culture that comprises only a single LAB species. Accordingly, in a preferred embodiment the LAB culture is a LAB culture selected from the group consisting of these cultures.

The LAB culture may be washed, or non-washed, before mixing with the protective agents.

Preferably, the LAB cell is a probiotic cell.

The frozen or dried cells can be mixed with any suitable excipients to make blends, for example human food and animal feed compositions.

The frozen or dried product comprising an asporogenous prokaryote, obtainable by the method described above, can be used to produce various types of compositions, wherein the potency of the bacteria is 1E+05 to 1E+12 CFU/g.

The compositions may be a food, feed, agricultural product, dietary supplement or pharmaceutical product.

The frozen or dried product comprising an asporogenous prokaryote, obtainable by the method described above, can also be used in methods of manufacturing a food, feed, agricultural product, dietary supplement or pharmaceutical product, said method comprising addition of a frozen or dried product.

### DESCRIPTION OF THE FIGURES

FIG. 1 shows the interfacial adhesion curves from BCSH assay for freeze-dried Bifidobacterium animalis subsp. lactis BB-12® without protectant (FD 0) and with protectant (FD 1). All data points are averages of three measurements with standard deviations.

FIG. 2 shows the interfacial adhesion curves from BCSH assay for freeze-dried Lactobacillus acidophilus LA-5® without protectant (FD 0) and with protectant (FD 1). All data points are averages of three measurements with standard deviations.

FIG. 3 shows the interfacial adhesion curves from BCSH assay for freeze-dried Lactobacillus reuteri RC-14® without protectant (FD 0) and with protectant (FD 1). All data points are averages of three measurements with standard deviations.

FIG. 4 shows the interfacial adhesion curves from BCSH assay for freeze-dried Lactobacillus animalis LA51 without protectant (FD 0) and with protectant (FD 1). All data points are averages of three measurements with standard deviations.

FIG. 5 shows the interfacial adhesion curves from BCSH assay for freeze-dried Streptococcus thermophilus TH-4® HA without protectant (FD 0) and with protectant (FD 1). All data points are averages of three measurements with standard deviations.

FIG. 6 shows the interfacial adhesion curves from BCSH assay for freeze-dried Lactococcus lactis R-607-1 without protectant (FD 0) and with protectant (FD 1). All data points are averages of three measurements with standard deviations.

FIG. 7. (A) relationship between loss of viabilities in the storage stability test at 37° C. and cell surface hydrophobicities of freeze-dried products without protectant. (B) relationship between loss of viabilities in the storage stability test at 37° C. and cell surface hydrophobicities of freeze-dried products with protectant.

### DEFINITIONS

Fructo-oligosaccharides (FOS), also known as oligofructose or oligofructan, are mixtures of oligosaccharide fructans. FOS can be produced by degradation of inulin, or polyfructose, a polymer of D-fructose residues linked by β(2→1) bonds with a terminal α(1→2) linked D-glucose. The degree of polymerization of inulin ranges from 10 to 60. Inulin can be degraded enzymatically or chemically to a mixture of oligosaccharides with the general structure Glu-Frun (abbrev. GFₙ) and Fruₘ (Fₘ), with n and m ranging from 1 to 7. This process also occurs to some extent in nature, and these oligosaccharides can be found in a large number of plants, especially in Jerusalem artichoke, chicory and the blue agave plant. The main components of commercial products are kestose (GF₂), nystose (GF₃), fructosylnystose (GF₄), bifurcose (GF₃), inulobiose (F₂), inulotriose (F₃), and inulotetraose (F₄). The second class of FOS is prepared by the transfructosylation action of a β-fructosidase of Aspergillus niger or Aspergillus on sucrose. The resulting mixture has the general formula of GFₙ, with n ranging from 1 to 5. Contrary to the inulin-derived FOS, as well as β(1→2) binding, other linkages do occur, however in limited numbers. In this patent application, “FOS” and cognate terms are used to describe the second class of FOS.

### EXAMPLES

The examples involve strains listed in Table 1. All strains have been deposited at a Depositary institution having acquired the status of international depositary authority under the Budapest Treaty on the International Recognition of the Deposit of Microorganisms for the Purposes of Patent Procedure: Leibniz Institute DSMZ-German Collection of Microorganisms and Cell Cultures Inhoffenstr. 7B, 38124 Braunschweig, Germany. The accession number given in Table 1.

The applicant requests that a sample of the deposited microorganisms stated below may only be made available to an expert, subject to available provisions governed by Industrial Property Offices of States Party to the Budapest Treaty, until the date on which the patent is granted.

TABLE 1
Overview of strains used in examples

| Strain | Accession No. | Deposit date |
|---|---|---|
| Bifidobacterium animalis subsp. | DSM 33868 | 2021 May 26 |
| lactis BB-12 ® | | |
| Lactobacillus acidophilus LA-5 ® | DSM 13241 | 2000 Jan. 20 |
| Lactobacillus animalis LA51 | DSM 33570 | 2020 Jul. 8 |
| Lactobacillus reuteri RC-14 ® | ATCC 55845 | 1996 Oct. 11 |
| Lactococcus lactis R-607-1 | DSM 21404 | 2008 Apr. 23 |
| Streptococcus thermophilus TH-4 ® HA | DSM 33869 | 2021 May 26 |

The recipe for protectant composition was adapted from the book Wolkers & Oldenhof (Eds), Cryopreservation and Freeze-drying Protocols, Third edition (2015) Chapter 24: Freeze-Drying of Lactic Acid Bacteria, Fernanda Fonseca, Stéphanie Cenard, and Stephanie Passot, p. 480, with following modification: 200 g/l sucrose was replaced by 150 g/l trehalose and 50 g/l gum arabic, 9 g/l NaCl was kept, and 5 g/l Na-ascorbate was increased to 10 g/l Na-ascorbate in demineralized water.

The various single protectants can for example be sourced as follows: glucose (dextrose monohydrate, Roquette Freres, France), lactose (lactose monohydrate, Arla Food Ingredients Group P/S, Denmark), Glucidex® IT12 (trade name of maltodextrin DE 12, Roquette Freres, France), fructooligosaccharides (FOS, Fructooligosaccharide 950P, Beghin-Meiji, France), trehalose (trehalose dihydrate, Cargill, Germany), inositol (Zhucheng, Haotian Pharmaceutical Co., Ltd., China), GENU® pectin YM-115-H (CP Kelco, Denmark) and gum arabic (Willy Benecke GmbH, Natural Gums, Germany). FOS is a mixture of saccharides with chain length varying between one and five saccharide units, 31-43 g GF2/100 g; 47-59 g GF3/100G and 4-16 g GF4/100 g FOS. Glucidex® IT 12 contains oligomers with 11-14 dextrose equivalents (97%); glucose (1%) and disaccharide (2%).

Carbohydrates were autoclaved for 20 min at 121° C. Sodium ascorbate was prepared by sterile-filtration and mixed with autoclaved carbohydrates immediately before use.

Enumeration of viable cells. Viable cell counts of Lactobacillus acidophilus LA-5®, Lactobacillus animalis LA51, Lactobacillus reuteri RC-14® were determined in duplicates by standard pour-plating method. The freeze-dried material was suspended in sterile peptone saline diluent and homogenized by stomaching. After 30 minutes of revitalization, stomaching was repeated and the cell suspension was serially diluted in peptone saline diluent. The dilutions were plated in duplicates on MRS agar (BD Difco™ Lactobacilli MRS Agar, Fisher Scientific). The agar plates were incubated anaerobically for three days at 37° C. Plates with 30-300 colonies were chosen for counting of colony forming units (CFU). The result was reported as average CFU/g freeze-dried sample, calculated from the quadruples.

Viable cells of Bifidobacterium animalis subsp. lactis BB-12® were determined by same method as for lactobacilli, except that the MRS agar (BD Difco™ Lactobacilli MRS Agar, Fisher Scientific) was supplemented with 0.5 g/l cysteine hydrochloride.

Viable cells of Streptococcus thermophilus TH-4® HA were determined similarly as for lactobacilli with following modifications: M-17 agar (Oxoid, England) was used instead of MRS agar. Incubation was conducted at 37° C. under aerobic conditions.

Viable cells of Lactococcus lactis R-607-1 were determined similarly as for lactobacilli with following modifications. M-17 agar (Oxoid, England) was used instead of MRS agar. Incubation was conducted at 30° C. under aerobic conditions.

Stability assessment. Stability of cells was assessed from the difference between CFU/g measured at the time 0 of the stability trial and at the specific sampling point of the stability test period. Loss of viability was quantified as a loss of log CFU/g.

Cell surface hydrophobicity was measured by the MATH method, and interfacial adhesion curves were determined. The method of Schär-Zammaretti & Ubbink ((2003) Biophysical Journal 85, 4076-4092)) was applied with modified buffer strength and the use of a cell wash in the initial step of the procedure: 0.2 g of freeze-dried granulate was resuspended in 10 ml of 100 mM sodium phosphate buffer (pH 7.0). The cell suspension was centrifuged at 5000 g for 10 minutes at a temperature 10° C. Supernatant was removed and cells were washed twice with the 100 mM sodium phosphate buffer. The washed cell pellet was resuspended in the 100 mM sodium phosphate buffer to optical density OD600 nm of 0.5±0.05. The suspension was mixed and aliquots of 3 ml were pipetted into plastic tubes. Hexadecane (99% purity, Sigma Aldrich) was added to the cell suspension in the following volumes: 10 μl, 30 μl, 100 μl, 200 μl, 400 μl, 800 μl, 1400 μl and 2000 μl hexadecane. Each combination of hexadecane and cell suspension in the buffer, Φ [V_{H}/V_{B}], was prepared in triplicate. The tubes were closed and the mixtures were vortexed one by one for 30 seconds at highest speed. Vortexing was repeated for 30 seconds once again for the whole sample series. The samples were left to rest for 5 minutes. 2 ml of aqueous phase was transferred to a cuvette for measurement of the OD₆₀₀ₙₘ. Bacterial cell surface hydrophobicity (BCSH) was calculated from the fraction of bacteria which adhered to the hexadecane/water interface according to the formula

$$BCSH ( \% ) = [ ( Initial OD 600 - Final OD 600 ) / Initial OD 600 ] * 100$$

A cell surface is classified as non-hydrophobic, i.e. hydrophilic, if partitioning of cells gives BCSH<20%. A hydrophobic cell surface is characterized by partitioning of cells with BCSH>50%, and a moderately hydrophobic surface has a BCSH in the range 20-50% (Lee and Yii (1996) Letters in Applied Microbiology 23: 343-346).

### Example 1

Bifidobacterium animalis subsp. lactis BB-12® was grown in MRS medium (BD Difco™ Lactobacilli MRS Broth, Fisher Scientific) supplemented with 0.5 g/l cysteine hydrochloride and 1 g/l sodium carbonate. Inoculum for fermentation was prepared by growing the strain in a closed bottle with the above specified growth medium, under static conditions and without pH control at 37° C. The incubation period was 16 hours. Fermentation was carried out in lab-scale fermenter, initiated by inoculation of 1% of pre-culture to the above specified MRS media. Fermentation was done under anaerobic conditions, with nitrogen in the headspace. The content of the fermenter was constantly stirred at 300 rpm, temperature was maintained at 37° C. and a pH set point 6.0 was controlled by addition of 24% ammonia water. Fermentation was completed in 16 hours, when all sugar was utilized by BB-12®. Fermentation broth was cooled down to 10° C. and concentrated by centrifugation at 4° C. The cell concentrate, prepared by concentration factor 30×, had dry matter of 14% (w/w). The cell concentrate was during further processing kept at temperature <10° C. The cell concentrate was divided in two aliquots: 1/ crude concentrate without any addition of protectant and 2/ cell concentrate for formulation with a protectant. The protectant contained 150 g/l trehalose (Trehalose dihydrate, Cargill, Germany), 50 g/l gum arabic (Willy Benecke GmbH, Germany), 9 g/l NaCl and 10 g/l Na-ascorbate in demineralized water. Dry matter of protectant was 22% (w/w). Protectant was added to the cell concentrate in such amount, that the ratio of the dry matter of protectant to the dry matter of cell concentrate was 1:1, i.e. 0.64 g of protectant with 22% dry matter (w/w) was dosed per 1 g of cell concentrate with dry matter 14% (w/w). After mixing of protectant with the cell concentrate, the formulated concentrate was within 15 minutes frozen by pelletization in liquid nitrogen. In parallel, crude concentrate was pelletized in liquid nitrogen. The frozen materials were freeze-dried to products BB-12® FD 0 and BB-12® FD 1, without the protectant and with the protectant, respectively.

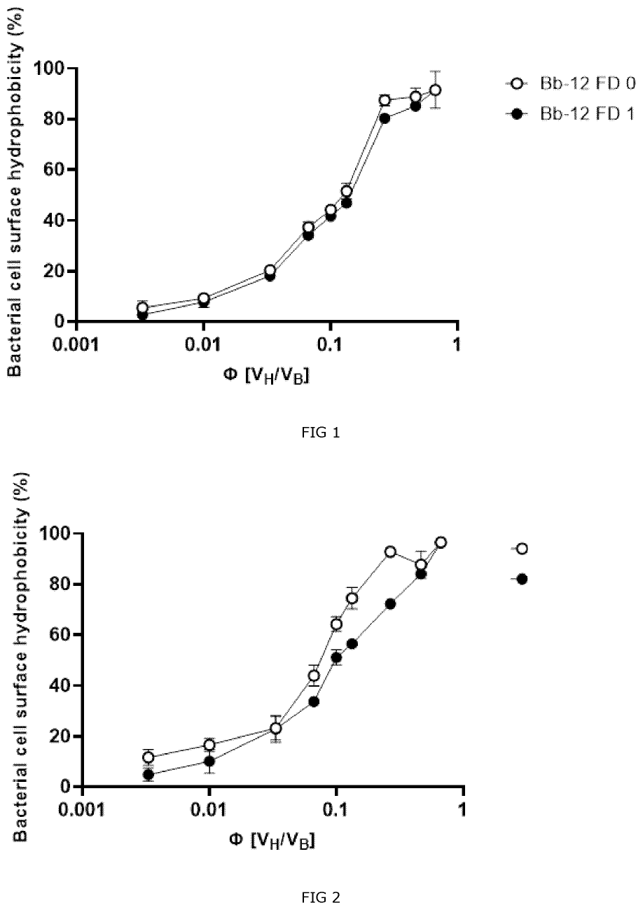

Cells from freeze-dried products BB-12® FD 0 and BB-12® FD 1 were characterized for cell surface hydrophobicity. Interfacial adhesion curves (FIG. 1) revealed that BB-12® had a highly stable composition of cell surface, not being affected by presence of the protectant. The cells exhibited increasing partitioning towards the hexadecane phase along with increasing volumes of hexadecane, what demonstrated presence of hydrophobic molecules on cell surfaces. At ϕ (V_{H}/V_{B})=0.1, the cell surface hydrophobicities of BB-12® FD 0 and BB-12® FD 1 were determined to be 44%±1% and 42%±2%, respectively. These values classify the BB-12® cell surface as moderate hydrophobic.

### Example 2

Lactobacillus acidophilus LA-5® was grown in MRS medium (BD Difco™ Lactobacilli MRS Broth, Fisher Scientific). Inoculum for fermentation was prepared by growing the strain in a closed bottle with the MRS medium, under static conditions and without pH control at 37° C. The incubation period was 16 hours. Fermentation was carried out in lab-scale fermenter, initiated by inoculation of 1% of pre-culture to the MRS media. Fermentation was done under anaerobic conditions, with nitrogen in the headspace. The content of the fermenter was constantly stirred at 300 rpm, temperature was maintained at 37° C. and a pH set point 5.5 was controlled by addition of 24% ammonia water. Fermentation was completed in 9 hours, when all sugar was utilized by LA-5®. Fermentation broth was cooled down to 10° C. and concentrated by centrifugation at 4° C. The cell concentrate, prepared by concentration factor 30×, had dry matter of 11% (w/w). The cell concentrate was further processed as described for BB-12® in Example 1. Formulation of LA-5® cell concentrate was done by dosing 0.5 g of protectant with 22% dry matter (w/w) per 1 g of LA-5® cell concentrate. Freeze-dried materials LA-5® FD 0 and LA-5® FD 1 were produced.

Cells of freeze-dried products LA-5® FD 0 and LA-5® FD 1 were characterized for cell surface hydrophobicity. Interfacial adhesion curves are compared in FIG. 2. Despite of a slight difference in the composition of surfaces of cells with and without protectant, both type of cells surfaces can be classified as hydrophobic, as the cell surface hydrophobicities at ϕ (V_{H}/V_{B})=0.1 were determined to be 64%±3% and 51%±3% for LA-5® FD 0 and LA-5® FD 1, respectively.

### Example 3

Lactobacillus reuteri RC-14® was grown in MRS medium (BD Difco™ Lactobacilli MRS Broth, Fisher Scientific). Inoculum for fermentation was prepared by growing the strain in a closed bottle with the MRS medium, under static conditions and without pH control at 37° C. The incubation period was 16 hours. Fermentation was carried out in lab-scale fermenter, initiated by inoculation of 1% of pre-culture to the MRS media. Fermentation was done under anaerobic conditions, with nitrogen in the headspace. The content of the fermenter was constantly stirred at 300 rpm, temperature was maintained at 37° C. and a pH set point 5.5 was controlled by addition of 24% ammonia water. Fermentation was completed in 9 hours, when all sugar was utilized by RC-14®. Fermentation broth was cooled down to 10° C. and concentrated by centrifugation at 4° C. The cell concentrate, prepared by concentration factor 30×, had a dry matter of 8.6% (w/w). The cell concentrate was further processed as described for BB-12® in Example 1. Formulation of RC-14® cell concentrate was done by dosing 0.39 g of protectant with 22% dry matter (w/w) per 1 g of RC-14® cell concentrate. Freeze-dried materials RC-14® FD 0 and RC-14® FD 1 were produced.

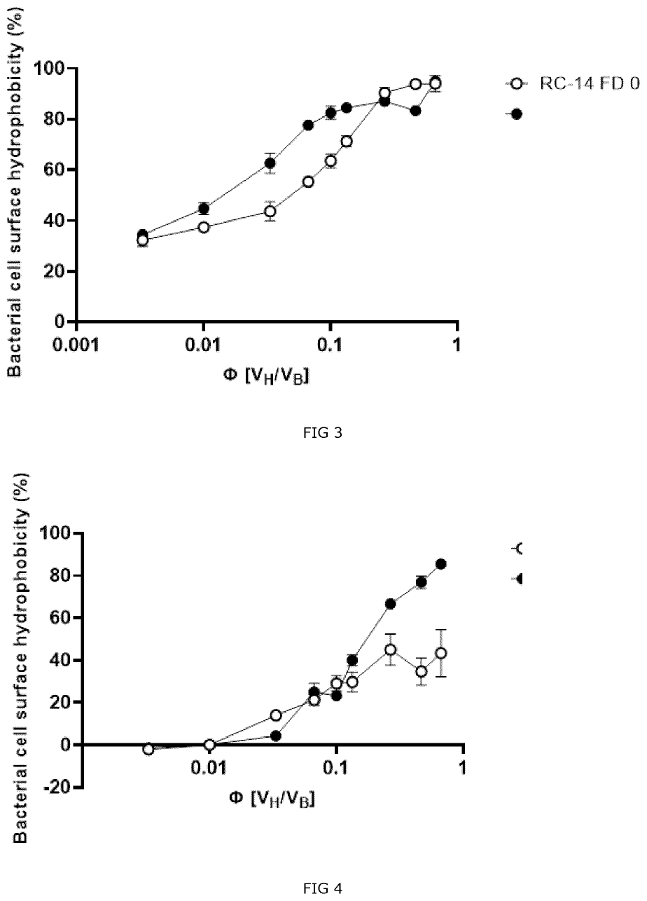

Cells of freeze-dried products RC-14® FD 0 and RC-14® FD 1 were characterized for cell surface hydrophobicity. Interfacial adhesion curves are shown in FIG. 3. The shape of curves reflected high degree of cells partitioning towards the hexadecane phase already for minimal volumes of hexadecane used. This finding correlated with high abundance of hydrophobic molecules. Use of protectant enhanced formation of hydrophobic structures further, as seen from increased response in the middle region of the interfacial adhesion curve. The cell surface hydrophobicities at ϕ (V_{H}/V_{B})=0.1 were determined to be 64%±3% and 83%±3% for RC-14® FD 0 and RC-14® FD 1, respectively. These values classify cell surface of RC-14® as highly hydrophobic.

### Example 4

Lactobacillus animalis LA-5® 1 was grown in MRS medium (BD Difco™ Lactobacilli MRS Broth, Fisher Scientific). Inoculum for fermentation was prepared by growing the strain in a closed bottle with the MRS medium, under static conditions and without pH control at 37° C. The incubation period was 7 hours. Fermentation was carried out in lab-scale fermenter, initiated by inoculation of 1% of pre-culture to the MRS media. Fermentation was done under anaerobic conditions, with nitrogen in the headspace. The content of the fermenter was constantly stirred at 300 rpm, temperature was maintained at 37° C. and a pH set point 5.5 was controlled by addition of 24% ammonia water. Fermentation was completed in 7 hours, when all sugar was utilized by LA51. Fermentation broth was cooled down to 10° C. and concentrated by centrifugation at 4° C. The cell concentrate, prepared by concentration factor 30×, had a dry matter of 13% (w/w). The cell concentrate was further processed as described for BB-12® in Example 1. Formulation of LA51 cell concentrate was done by dosing 0.6 g of protectant with 22% dry matter (w/w) per 1 g of LA51 cell concentrate. Freeze-dried materials LA51 FD 0 and LA51 FD 1 were produced.

Cells of freeze-dried products LA51 FD 0 and LA51 FD 1 were characterized for cell surface hydrophobicity. Interfacial adhesion curves are shown in FIG. 4. The shape of curve for LA51 FD 0 reflected weaker partitioning of cells towards the hexadecane, what correlated with reduced abundance of hydrophobic molecules on cell surface. Presence of protectant modulated LA-5® 1 cell surface towards increased hydrophobicity, as seen by higher hydrophobic response of LA51 FD 1 cells atϕ (V_{H}/V_{B}) >0.1. The cell surface hydrophobicities atϕ (V_{H}/V_{B})=0.1 were determined to be 29%±4% and 23%±2% for LA51 FD 0 and LA51 FD 1, respectively. These values classify cell surface of LA51 as moderate hydrophobic.

### Example 5

Streptococcus thermophilus TH-4® HA was grown in MRS medium (BD Difco™ Lactobacilli MRS Broth, Fisher Scientific). Inoculum for fermentation was prepared by growing the strain in a closed bottle with the MRS medium, under static conditions and without pH control at 40° C. The incubation period was 16 hours. Fermentation was carried out in lab-scale fermenter, initiated by inoculation of 1% of pre-culture to the MRS media. Fermentation was done under anaerobic conditions, with nitrogen in the headspace. The content of the fermenter was constantly stirred at 300 rpm, temperature was maintained at 40° C. and a pH set point 6.0 was controlled by addition of 24% ammonia water. Fermentation was completed in 18 hours, when all sugar was utilized by TH-4® HA. Fermentation broth was cooled down to 10° C. and concentrated by centrifugation at 4° C. The cell concentrate, prepared by concentration factor 30×, had a dry matter of 7.8% (w/w). The cell concentrate was further processed as described for BB-12® in Example 1. Formulation of TH-4® HA cell concentrate was done by dosing 0.35 g of protectant with 22% dry matter (w/w) per 1 g of TH-4® HA cell concentrate. Freeze-dried materials TH-4® HA FD 0 and TH-4® HA FD 1 were produced.

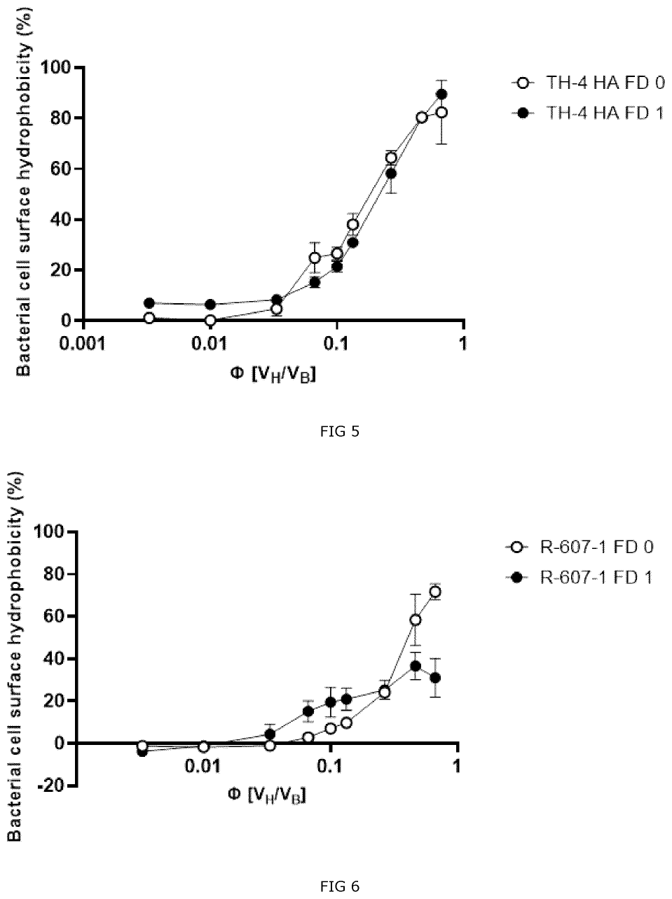

Cells of freeze-dried products TH-4® HA FD 0 and TH-4® HA FD 1 were characterized for cell surface hydrophobicity. Interfacial adhesion curves are shown in FIG. 5. The cell surface properties of freeze-dried cells without and with protectant were similar.

The hydrophobicities at ϕ (V_{H}/V_{B})=0.1 were determined to be 27%±3% and 22%±2% for TH-4® HA FD 0 and TH-4® HA FD 1, respectively. These values classify cell surface of TH-4® HA as moderate hydrophobic.

### Example 6

Lactococcus lactis R-607-1 was grown in Difco™ M-17 broth (Becton, Dickinson and Company, USA) modified with respect to the concentration of lactose. Lactose concentration was increased to 20 g/l. Inoculum for fermentation was prepared by growing the strain in a closed bottle with the modified M-17 medium, under static conditions and without pH control at 30° C. The incubation period was 16 hours. Fermentation was carried out in lab-scale fermenter, initiated by inoculation of 1% of pre-culture to the modified M-17 media. Fermentation was done under anaerobic conditions, with nitrogen in the headspace. Content of the fermenter was constantly stirred at 300 rpm, temperature was maintained at 30° C. and a pH set point 6.0 was controlled by addition of 24% ammonia water. Fermentation was completed in 8 hours, when all sugar was utilized by R-607-1. Fermentation broth was cooled down to 10° C. and concentrated by centrifugation at 4° C. The cell concentrate, prepared by concentration factor 30×, had a dry matter of 12.5% (w/w). The cell concentrate was further processed as described for BB-12® in Example 1. Formulation of R-607-1 cell concentrate was done by dosing 0.57 g of protectant with 22% dry matter (w/w) per 1 g of R-607-1 TH-4® HA cell concentrate. Freeze-dried materials R-607-1 FD 0 and R-607-1 FD 1 were produced.

Cells of freeze-dried products R-607-1 FD 0 and R-607-1 FD 1 were characterized for cell surface hydrophobicity. Interfacial adhesion curves are shown in FIG. 6. The shape of curve reflected lower degree of cells partitioning towards the hexadecane. Formulation of cells with protectant modified the hydrophobic structures on cell surface. The cell surface hydrophobicities at ϕ (V_{H}/V_{B})=0.1 were determined to be 7%±2% and 19%±7% for R-607-1 FD 0 and R-607-1 FD 1, respectively. These values classify cell surface of R-607-1 as non-hydrophobic.

### Example 7

Freeze-dried granulates FD 0 and FD 1, which were produced in Example 1-Example 6, were distributed in aliquots in aluminum bags, the bags were sealed and subjected to accelerated storage stability tests at 37° C. Survival of cells was determined after 2 weeks of incubation at 37° C., the results are summarized in Table 2.

TABLE 2
CFU counts from 37° C. storage stability study with freeze-dried products
of bifidobacteria, lactobacilli, streptococci and lactococci.

| | log CFU/g | | |
|---|---|---|---|
| FD product | 0 weeks | 2 weeks | Δ log CFU/g |
| BB-12 ® FD 0 | 11.66 | 8.32 | 3.34 |
| BB-12 ® FD 1 | 11.29 | 11.17 | 0.12 |
| LA-5 ® FD 0 | 10.89 | 8.29 | 2.60 |
| LA-5 ® FD 1 | 11.00 | 10.55 | 0.45 |
| RC-14 ® FD 0 | 11.48 | 9.68 | 1.80 |
| RC-14 ® FD 1 | 11.1 | 10.85 | 0.25 |
| LA51 FD 0 | 11.91 | 11.14 | 0.77 |
| LA51 FD 1 | 11.71 | 11.36 | 0.35 |
| R-607-1 FD 0 | 12.13 | 10.98 | 1.15 |
| R-607-1 FD 1 | 11.93 | 11.54 | 0.39 |
| TH-4 ® HA FD 0 | 10.89 | 9.62 | 1.27 |
| TH-4 ® HA FD 1 | 10.80 | 10.06 | 0.74 |

In general, freeze-dried products FD 0, without any protectant showed poor stabilities in comparison to freeze-dried products FD 1, which contained protectants.

A correlation between loss of viabilities in the storage stability test and cells surface hydrophobicities of the six representants of lactic acid bacteria was investigated.

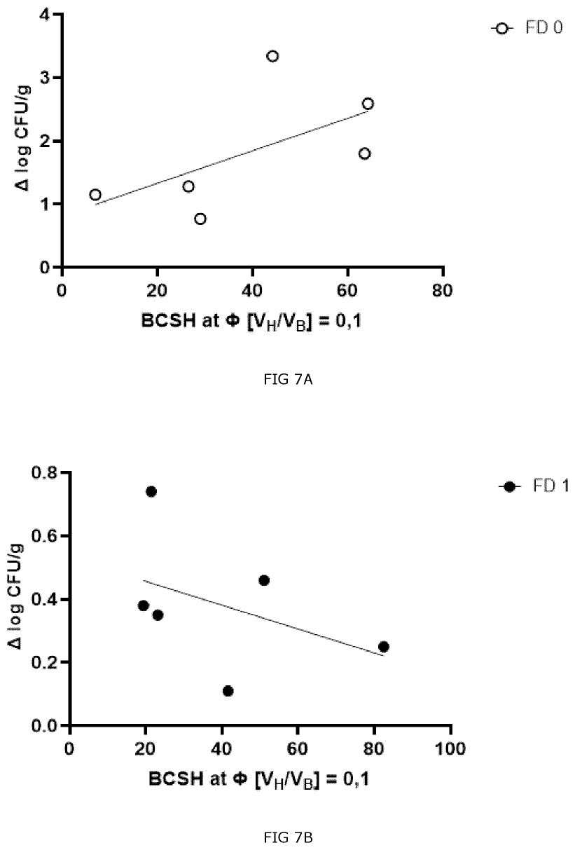

FIGS. 7A and 7B show correlations between logarithmic loss of CFU/g FD products (cf. Table 2) and surface hydrophobicity of freeze-dried cells, at ϕ (V_{H}/V_{B})=0.1, in freeze-dried products without protectant and with protectant, respectively. These values have been specified in Example 1 to Example 6.

FIG. 7A: loss of storage stability in the group of FD products without protectant, FD 0 products, was found to be directly correlated to the cell surface hydrophobicity. The equation of linear regression was as follows: y=0.0257x+0.8173; R²=0.3545. The freeze-dried, non-protected strains with more hydrophobic cell surface were found to be less stable than the freeze-dried, non-protected strains with lower cell surface hydrophobicity.

FIG. 7B: analysis of the trend for freeze-dried products with protectant, FD 1 products, showed, surprisingly, the opposite trend. The equation for linear regressions was as follows: y=−0.003768x+0.5319; R²=0.1867. Loss of storage stability in the group of FD 1 products, was found to be inversely correlated with the cell surface hydrophobicity. The protectant-containing, freeze-dried strains with more hydrophobic cell surface exhibited higher storage stability than strains with lower cell surface hydrophobicity.

1. A method of preparing a frozen or dried product comprising asporogenous prokaryote cells, the method comprising the steps of:
i. growing the prokaryote cells by fermentation in a fermentation broth;
ii. concentrating the cells by separation of the fermentation broth from the cells to produce a cell concentrate;
iii. combining cell concentrate with a medium containing a protective compound to obtain a preprocessing composition;
iv. holding the preprocessing composition under conditions not inducing metabolic activity; and
v. preserving the composition by:
(a) freezing the composition to form a frozen prokaryote product;
(b) drying the product to form a dried prokaryote product; or
(c) freezing the composition to form a frozen prokaryote intermediate product and then lyophilising the intermediate product to form a freeze-dried prokaryote product,
wherein the hydrophobicity of the cell surface of the cells is in the product is at least 40%, as measured by the MATH method at 22° C. and expressed as [(Initial OD₆₀₀−Final OD₆₀₀)/Initial OD₆₀₀]*100 when measured with a ϕ [V_{H}/V_{B}] at at least one point between 0.1 and 1.0 and the initial OD₆₀₀ (nm) is 0.5 and wherein the potency of product is 1E+08 to 1E+13 CFU/g.

2. The method according to claim 1, wherein the protective compound is one or more of:
a monosaccharide;
a disaccharide;
a sugar alcohol;
a trisaccharide;
an oligosaccharide; and
a polysaccharide.

3. The method according to claim 1, wherein the frozen prokaryote product or the frozen prokaryote intermediate product has a dry weight ratio of the final protectant to cell concentrate of between 10:1 and 0.1:1.

4. The method according to claim 1, wherein step (iii) lasts for 0.25 to 16 hours.

5. The method according to claim 1, wherein step (iii) is carried out at 4° C. to 20° C.

6. The method according to claim 1, wherein the prokaryote is a fermentative bacterium selected from the phylum Firmicutes, Actinobacteria, Bacteroidetes, and Verrucomicrobia.

7. The method according to claim 6, wherein the prokaryote is one or more of: Limosilactobacillus reuteri, Lacticaseibacillus rhamnosus, Ligilactobacillus salivarius, Lacticaseibacillus casei, Lacticaseibacillus paracasei subsp. paracasei, Lactiplantibacillus plantarum subsp. plantarum, Limosilactobacillus fermentum, Ligilactobacillus animalis, Lentilactobacillus buchneri, Latilactobacillus curvatus, Companilactobacillus futsaii, Latilactobacillus sakei subsp. sakei, Lactiplantibacillus pentosus, Levilactobacillus brevis, Lactobacillus delbrueckii subsp. bulgaricus, Lactobacillus delbrueckii subsp. lactis, Lactobacillus gasseri, Lactobacillus johnsonii, Lactobacillus helveticus and Lactobacillus acidophilus, Lactobacillus jensenii, and Lactobacillus iners.

8. A frozen or dried product comprising an asporogenous prokaryote obtained by the method according to claim 1.

9. The frozen or dried product according to claim 8, wherein the potency of the product is 1E+08 to 1E+13 CFU/g.

10. A composition comprising the frozen or dried product according to claim 8, wherein potency of the bacteria is 1E+05 to 1E+12 CFU/g.

11. The composition according to claim 10, which is a food, feed, agricultural product, dietary supplement or pharmaceutical product.

12. (canceled)

13. The method according to claim 1, wherein the frozen prokaryote product or the frozen prokaryote intermediate product has a dry weight ratio of the final protectant to cell concentrate of between 3:1 and 0.5:1.

14. The method according to claim 1, wherein the frozen prokaryote product or the frozen prokaryote intermediate product has a dry weight ratio of the final protectant to cell concentrate of between 2:1 and 1:1.

15. The method according to claim 1, wherein step (iii) is carried out below 10° C.
